(19) United States
(12) Patent Application Publication
(10) Pub. No.: US 2026/0218266 A1
(43) Pub. Date: Jul. 30, 2026

(54) CELL CULTURE METHOD TO INCREASE CELL BIOMASS, PRODUCTIVITY AND PROTEIN TITER

(71) Applicant: KASHIV BIOSCIENCES, LLC, Piscataway, NJ (US)

(72) Inventors: Akhilesh Kushwaha, Ahmedabad (IN); Sudharti Gupta, Ahmedabad (IN); Chandramauli Rawal, Piscataway, NJ (US); Kaushal Joshi, Ahmedabad (IN)

(21) Appl. No.: 19/157,902

(22) PCT Filed: Feb. 20, 2024
(86) PCT No.: PCT/IB2024/051621

§ 371 Date: Aug. 19, 2025

(30) Foreign Application Priority Data
Feb. 20, 2023 (IN) 202321011404

(51) Int. Cl. C12P 21/00; C12M 1/00; C12N 1/20

(52) U.S. Cl. CPC C12P 21/00; C12M 23/58; C12N 1/20; C07K 2317/14

(57) ABSTRACT

The method disclosed in the current invention is used to increase cell biomass, productivity and protein titer in cell culture medium by using at least more than one fermenter. The method specifically discloses cell culture conditions used in fermenter. More specifically, the method discloses the use of primary and secondary fermenter wherein the primary fermenter comprises different cell culture conditions compared to secondary fermenter.

### FIELD OF THE INVENTION

The method disclosed in the current invention is used to increase cell biomass, productivity and protein titer in cell culture medium by using at least more than one fermenter. The method specifically discloses cell culture conditions used in fermenter. More specifically, the method discloses the use of primary and secondary fermenter wherein the primary fermenter comprises different cell culture conditions compared to secondary fermenter.

### BACKGROUND OF THE INVENTION

Current ongoing efforts to maximize bioreactor or fermenter productivity in both time and volume directly affect the scale and capital investment required for a bioreactor suite.

Recombinantly produced protein products are increasingly becoming medically and clinically important for use as therapeutics, treatments, and prophylactics. Therefore, the development of reliable cell culture processes that economically and efficiently achieve an increased cell density thereby resulting in increased final protein product concentration fulfills both a desired and needed goal in the art.

Hence, there is a need for an improved process to achieve higher level of protein titer in a short duration.

In the present invention, increased cell density or cell biomass, productivity and protein titer has been achieved to a satisfactory level compared to the conventional method.

### SUMMARY OF THE INVENTION

In an embodiment, a cell culture process for production of a recombinant protein, comprising culturing the cells in primary and secondary fermenter and/or bioreactor wherein the primary and secondary fermenter and/or bioreactor is connected to each other to perform transfer of cultured cells from primary fermenter and/or bioreactor to secondary fermenter and/or bioreactor.

In an embodiment, a cell culture process for production of a recombinant protein, comprising culturing the cells in primary fermenter and/or bioreactor to obtain suitable cell biomass and transferred the cell biomass to secondary fermenter and/or bioreactor for protein production.

In an embodiment, the invention provides a cell culture process for production of a recombinant protein, comprising:
a) culturing cells in a primary fermenter which produces a desired amount of biomass in cell culture under conditions that allow for protein production;
b) feeding the cells in the primary fermenter with feeding medium containing glucose;
c) transferring the cultured cells from the primary fermenter into a secondary fermenter which is connected to each other; and
d) expressing the recombinant protein in the secondary fermenter;
wherein transferring of the cultured cells into the secondary fermenter is performed when biomass level is about 150 g/L to about 230 g/L.

In an embodiment, the invention provides a cell culture process for production of protein comprising:
a) culturing cells in a primary fermenter to achieve desired biomass (g/L);
b) transferring the cultured cells in a secondary fermenter to obtain desired yield of protein of interest; and
c) expressing the protein of interest in the secondary fermenter;
wherein the process provides improved yield compared to the process performed in single fermenter.

In an embodiment, feeding is performed in primary fermenter, wherein the feeding is exponential feeding.

In another embodiment, feeding is performed in primary fermenter is constant feeding.

In an embodiment, feeding is performed in secondary fermenter, wherein the feeding is exponential feeding.

In another embodiment, feeding is performed in secondary fermenter is constant feeding.

In an embodiment, the constant feeding is about 3 g/L to about 5 g/L in the secondary fermenter.

In an embodiment, feeding is performed in primary and secondary fermenter is different.

In an embodiment, the first exponential feeding is about 10±2% in an incremental order at every hour, wherein the exponential feeding is occurring in the primary fermenter.

In an embodiment, feeding is performed every hour from the beginning of the cell culture.

In an embodiment, the first feeding is started in the primary fermenter after culturing the cells for about 3 hours to 4 hours.

In an embodiment, the feeding of cell culture is performed continuously or intermittently.

In an embodiment, the exponential feeding in the primary fermenter is performed at or about 4 hours after the cell culture.

In an embodiment, the constant feeding is performed in the secondary fermenter from beginning of the cell culture.

In an embodiment, transferring of the cell culture from the primary fermenter to the secondary fermenter occurs once the cell culture of the primary fermenter achieved stationary phase.

In an embodiment, transferring of the cell culture from the primary fermenter to the secondary fermenter occurs once the biomass is about 150 g/L to about 230 g/L.

In an embodiment, transferring of the cell culture from the primary fermenter to the secondary fermenter occurs once the biomass is about 170 g/L to about 200 g/L.

In an embodiment, transferring of the cell culture from the primary fermenter to the secondary fermenter occurs once the cell culture of the primary fermenter achieved OD600 is about 120 to about OD600 140.

In an embodiment, transferring of the cell culture broth from the primary fermenter to the secondary fermenter is about 50% to about 100%.

In an embodiment, the total amount of cell culture broth transferred from the primary fermenter to the secondary fermenter is about 90 to about 95%.

In an embodiment, induction is performed in the secondary fermenter immediately after the cell culture broth has been transferred from the primary fermenter.

In another embodiment, induction is performed in the secondary fermenter for about 8 hours to about 14 hours.

In another embodiment, induction is performed in the secondary fermenter for about 10 hours to about 12 hours.

In an embodiment, induction in the secondary fermenter is performed either in ramp up, ramp down, constant or in bolus mode.

In an embodiment, harvesting the protein from the liquid cell culture, wherein the harvesting occurs after completion of the induction phase.

In an embodiment, the invention provides a cell culture process for production of protein comprising:
a) culturing cells in a primary fermenter;
b) feeding glucose in the primary fermenter under suitable conditions;
c) transferring the cells from the primary fermenter to a secondary fermenter to obtain desired yield of protein of interest; and
d) expressing the protein of interest in the secondary fermenter;
wherein feeding of glucose is performed at suitable time during the culture in the primary fermenter, increased the cell density in primary fermenter which leads to an improve protein titer in the secondary fermenter; wherein the induction is performed in secondary fermenter to express and harvest the protein of interest from the secondary fermenter.

In an embodiment, the invention provides a cell culture process for production of protein comprising:
a) culturing cells in the primary fermenter;
b) feeding glucose in a primary fermenter under suitable conditions;
c) transferring the cells from the primary fermenter to a secondary fermenter to obtain desired yield of protein of interest; and
d) expressing the protein of interest in the secondary fermenter;
wherein feeding of glucose is performed at suitable time during the culture in the primary fermenter, increased the cell density in primary fermenter and improved protein titer in secondary fermenter compared to feeding is performed at the beginning of culture in primary fermenter; wherein the induction is performed in secondary fermenter to express and harvest the protein of interest from the secondary fermenter.

In an embodiment, the invention provides a cell culture process for production of protein comprising:
a) culturing cells in a primary fermenter;
b) feeding glucose in the primary fermenter under suitable conditions;
c) transferring the cells from the primary fermenter to a secondary fermenter to obtain desired yield of protein of interest; and
d) expressing the protein of interest in the secondary fermenter;
wherein feeding of glucose is performed at suitable time during the culture in the primary fermenter to achieve cell density at least OD600 120 to 140; wherein the induction is performed in secondary fermenter to express and harvest the protein of interest from the secondary fermenter.

In an embodiment, the feeding of glucose is not performed in the secondary fermenter.

In an embodiment, the feeding of glucose is performed in the primary fermenter after culturing the cells for about 3 hours to about 4 hours.

In an embodiment, the feeding of glucose is performed in the primary fermenter at about 7 hours to about 8 hours when cell culture is performed for about 12 hours.

In an embodiment, the invention provides a cell culture process for production of recombinant protein comprising:
a) culturing cells in a serum free, chemically defined complex media, wherein the culturing of cells from a seed culture to a first liquid cell culture;
b) expanding the cells that produce recombinant protein, wherein expanding of the cell culture is from the first liquid cell culture to a second liquid cell culture; and
c) harvesting the recombinant protein from the second liquid cell culture;
wherein the first liquid cell culture is transferred to the second liquid cell culture after achieving biomass about 150 g/l to about 230 g/l.

In an embodiment, a process for cell culture to produce a recombinant protein, cell culture process comprising:
a) culturing cells in a primary fermenter which produces a desired amount of biomass in cell culture under conditions that allow for protein production;
b) feeding the cells in the primary fermenter with feeding medium containing glucose;
c) transferring the cultured cells from the primary fermenter into a secondary fermenter which is connected to each other; and
d) feeding the cells in the secondary fermenter with feeding medium;
e) expressing the recombinant protein in the secondary fermenter;
wherein transferring of the cultured cells into the secondary fermenter is performed when biomass level is about 150 g/L to about 230 g/L; wherein the cell density, cell biomass, titer and total inclusion bodies are increased.

In an embodiment, a method for increasing titer of a recombinant protein in a cell culture process comprising:
a) culturing cells in a primary fermenter which produces a desired amount of biomass in cell culture under conditions that allow for protein production;
b) feeding the cells in the primary fermenter with feeding medium containing glucose;
c) transferring the cultured cells from the primary fermenter into a secondary fermenter which is connected to each other; and
d) feeding the cells in the secondary fermenter with feeding medium;
e) expressing the recombinant protein in the secondary fermenter;
wherein transferring of the cultured cells into the secondary fermenter is performed when biomass level is about 150 g/L to about 230 g/L.

In an embodiment, the liquid culture can be at least of 200 L, 500 L, 1000 L, 2000 L or more.

In an embodiment, culturing the first liquid cell culture with a seed density is about 3 to 5 OD600 with a variation of 5-10% V/V inoculum.

In an embodiment, the pH is maintained during the cell culture process is from about 6.8 to about 7.2, preferably 7.0.

In an embodiment, the dissolved oxygen (DO) is maintained during the cell culture process is from about 20% to 70%, preferably 30%.

In an embodiment, the temperature is maintained during the cell culture process is from about 35.5° C. to about 38.5° C., preferably 37° C.

In an embodiment, the method discloses the use of primary and secondary fermenter wherein the primary fermenter comprises different cell culture conditions compared to secondary fermenter.

In an embodiment, the inducer used in the secondary fermenter is selected from IPTG and arabinose.

In an embodiment, the cells are cultured for about 8 hours to about 14 hours in the secondary fermenter.

In an embodiment, the cells are cultured for about 10 hours to about 12 hours in the secondary fermenter.

In an embodiment, amount of inclusion bodies obtained from the cell culture is about 15 g/L to about 30 g/L.

In an embodiment, protein titer is obtained from the secondary cell culture is about 4 g/L to about 8 g/L.

In an embodiment, the production process is performed continuously for about 3 days to about 5 days.

In an embodiment, the cell culture media is non-animal origin.

In an embodiment, the cells that produces recombinant proteins in semi continuous large-scale fermenter are microbial cells.

In an embodiment, the cells that used for producing recombinant proteins in cell culture medium are E. coli cells.

In an embodiment, the recombinant protein produced by E. coli is a fragmented antibody.

In an embodiment, the recombinant protein produced by E. coli is Certolizumab pegol.

In an embodiment, the feeding medium containing glucose is added at concentration of about 10 g/l to about 20 g/L.

### BRIEF DESCRIPTION OF FIGURE

FIG. 1: depicts the schematic representation of two fermenter.

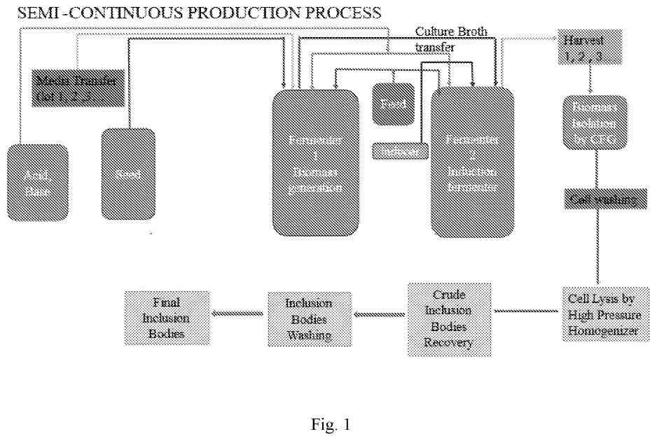

### DETAIL DESCRIPTION OF THE INVENTION

The term “comprises” or “comprising” is used in the present description, it does not exclude other elements or steps. For the purpose of the present invention, the term “consisting of” is considered to be an optional embodiment of the term “comprising of”. If hereinafter a group is defined to comprise at least a certain number of embodiments, this is also to be understood to disclose a group which optionally consists only of these embodiments.

As used throughout the specification and in the appended claims, the singular forms “a”, “an”, and “the” include the plural reference unless the context clearly dictates otherwise.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

The term “feed” as used herein refers to any addition of any substance made to a cell culture after inoculation. Feeding can be one or more additions.

The term “exponential feeding” as used herein refers to addition of any substance made to a cell culture after inoculation in an increasing order. For example, feeding amount at first hour is 1×, at second hour will be 2× or more, third hour will be 3× or more.

The term “constant feeding” as used herein refers to addition of any substance made to a cell culture after inoculation in an equal amount of feed throughout the cell culture. For example, feeding amount at first hour is 1×, at second hour & third hour will be also 1× and same throughout the process.

The term “inoculation” as used herein refers to the addition of cells to starting medium to begin the culture.

The term “feed medium”, “feed media” and “feeding medium” as used herein refers to a medium containing one or more nutrients that is added to the culture beginning at some time after inoculation.

The term “basal medium” and “basal media” as used herein refers to starting medium to which cells are added to begin the culture.

The term “liquid culture” as used herein refers to cells (for example, bacteria, plant, insect, yeast, or animal cells) grown on supports, or growing suspended in a liquid nutrient medium.

The term “seed culture” as used herein refers to a cell culture grown in order to be used to inoculate larger volumes of culture medium. The seed culture can be used to inoculate larger volumes of media in order to expand the number of cells growing in the culture (for example, cells grown in suspension).

The term “culturing” as used herein refers to growing one or more cells in vitro under defined or controlled conditions. Examples of culturing conditions which can be defined include temperature, gas mixture, time, and medium formulation.

The term “fed-batch culture” or “semi continuous cell culture” as used herein refers to a method of culturing cells in which additional components are provided to the culture at some time subsequent to the beginning of the culture process. The provided components typically comprise nutritional supplements for the cells that have been depleted during the culturing process. A fed-batch culture is typically stopped at some point and the cells and/or components in the medium are harvested and optionally purified. The term “semi-continuous large-scale fermenter” or “fed-batch cell culture fermenter” as used herein are interchangeable.

The term “expanding” as used herein refers to culturing one or more cells in vitro for the purpose of obtaining a larger number of cells in the culture.

The term “growth phase” as used herein of the cell culture as used herein refers to the period of exponential cell growth (for example, the log phase) where cells are primarily dividing rapidly. During this phase, the rate of increase in the density of viable cells is higher than at any other time point.

The term “production phase” of the cell culture as used herein refers to the period of time during which cell growth is stationary or is maintained at a near constant level. The density of viable cells remains approximately constant over a given period of time. Logarithmic cell growth has terminated, and protein production is the primary activity during the production phase. The medium at this time is generally supplemented to support continued protein production and to achieve the desired glycoprotein product.

The term “cell culture condition” as used herein refers to the conditions suitable for improving cell biomass and protein titer. In an embodiment, cell culture conditions are selected from basal media, feed media, feeding including constant and exponential, temperature, pH, duration of culture, dissolved oxygen, CO₂.

In an embodiment, cell culture conditions maintained during primary fermenter is different than secondary fermenter. In an embodiment, cell culture conditions maintain during primary fermenter to improve the biomass. In an embodiment, cell culture conditions maintain during primary fermenter to improve the biomass by achieving OD600 is about 120 to about 130. In an embodiment, primary fermenter maintains exponential feeding.

In an embodiment, cell culture conditions maintained during secondary fermenter is different than primary fermenter. In an embodiment, cell culture conditions maintain during secondary fermenter to increase the protein titer. In an embodiment, cell culture conditions maintain during primary fermenter to improve the protein titer in secondary fermenter is about 4 g/l to about 8 g/l. In an embodiment, secondary fermenter maintains constant feeding.

The term “semi continuous process” as used herein refers to the cell culture process comprising at least two fermenters connected to each other where the first fermenter and/or primary fermenter used for biomass generation and the second fermenter and/or secondary fermenter used for expression of protein of interest.

The term “primary fermenter” as used herein refers to the cell culture fermenter used for only biomass generation. In certain embodiment, primary fermenter is performed after the seed culture. In an embodiment, the feeding medium contains glucose. In an embodiment, feeding medium contains glucose at a concentration of about 10 g/l to about 20 g/L. In an embodiment, primary fermenter may contain one or more fermenter. In an embodiment, primary fermenter is performed for at least 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours. In an embodiment, the primary fermenter comprises continuously feeding on every hour. In an embodiment, the primary fermenter comprises intermittent feeding at suitable hours. In an embodiment, intermittent feeding is performed in suitable interval of 10 minutes or 20 minutes or 30 minutes or 40 minutes or 1 hour or 2 hours or 3 hours or 4 hours or 5 hours or in continuous mode. For commercial purpose, the primary fermenter is performed from at least more than 5 hours. It is well understood to skilled person that feeding can be adjusted based on the duration of primary fermenter. In certain embodiment, feeding can be initiated from each hour till harvest or before the harvest. For an example, if primary fermenter is operated for about 10 hours to about 12 hours, then feeding is performed from about 3 hours to about 4 hours of the beginning of cell culture in primary fermenter. In another embodiment, if primary fermenter is operated for about 12 hours to about 16 hours, then feeding is performed from about 5 hours to about 6 hours of the beginning of cell culture in the primary fermenter. In certain embodiment, the feeding in the primary fermenter is performed from the beginning of the cell culture.

The term “secondary fermenter” as used herein refers to the cell culture fermenter used for expression of protein of interest. The cell culture in the secondary fermenter is obtained from the primary fermenter. In an embodiment, the feeding in the secondary fermenter is constant feeding. In an embodiment, the feeding in the secondary fermenter is intermittent. In an embodiment, the feeding medium does not contain glucose. In an embodiment, the cells are culturing in the secondary fermenter for about 6 hours to about 16 hours. In an embodiment, the cells are culturing in the secondary fermenter for about 8 hours to about 14 hours. In an embodiment, the cells are culturing in the secondary fermenter for about 10 hours to about 12 hours. In another embodiment, induction starts in the secondary fermenter from the beginning of the cell culture. In an embodiment, the induction starts in the secondary fermenter from the beginning of the cell culture until the cell culturing ends. In an embodiment, inducer used for induction is selected from IPTG and arabinose.

The term “Productivity” as used herein refers to the overall cell culture process where the outputs of the process is increased i.e., yield, titer, overall process time, cell biomass, cell density etc.

The term “cell density” as used herein refers to the number of cells present in a given volume of medium.

The term “titer” or “protein titer” or “protein expression” are interchangeable and used herein refers to the total amount of polypeptide of interest produced by a cell culture (e.g., an animal cell culture or bacterial cell culture), divided by a given amount of medium volume; thus “titer” refers to a concentration. Titer is typically expressed in units of milligrams of polypeptide per liter of medium. The modified fed-batch culture of the present invention has an effect of increasing polypeptide product titer compared to other cell culture methods known in the art.

The term “biomass generation” as used herein refers to growing of cells with a higher number or high cell density.

The term “inducing” or “induction” as used herein are interchangeable refers to the addition of inducing agent such as IPTG, arabinose in to the second cell culture fermenter for expression of proteins.

The term “harvest” or “harvesting” as used herein are interchangeable refers to the process of removal of cells, cell debris, and other soluble and insoluble impurities from the culture media prior to sterile filtration and purification.

The term “Inclusion body” as used herein is an amorphous deposit in the cytoplasm of a cell; an aggregated protein appropriate to the cell but damaged, improperly folded or liganded, or a similarly inappropriately processed foreign protein, such as a viral coat protein or recombinant DNA product.

The term “ramp up” as used herein refers to addition of substance in an increasing order.

The term “ramp down” as used herein refers to addition of substance in a decreasing order.

The term “bolus” or “bolus mode” as used herein refers to addition of substance in a single shot.

The term “constant” as used herein refers to the addition of substance at same concentration or same amount throughout the whole process.

In an embodiment, the invention provides a cell culture process for production of a recombinant protein, comprising:
a) culturing cells in a primary fermenter which produces a desired amount of biomass in cell culture under conditions that allow for protein production;
b) feeding the cells in the primary fermenter with feeding medium containing glucose;
c) transferring the cultured cells from the primary fermenter into a secondary fermenter which is connected to each other; and
d) expressing the recombinant protein in the secondary fermenter;
wherein transferring of the cultured cells into the secondary fermenter is performed when biomass level is about 150 g/L to about 230 g/L.

In an embodiment, the invention provides a cell culture process for production of protein comprising:
a) culturing cells in a primary fermenter to achieve desired biomass (g/L);
b) transferring the cultured cells in a secondary fermenter to obtain desired yield of protein of interest; and
c) expressing the protein of interest in the secondary fermenter;
wherein the process provides improved yield compared to the process performed in single fermenter.

In an embodiment, feeding is performed in primary fermenter, wherein the feeding is exponential feeding.

In another embodiment, feeding is performed in primary fermenter is constant feeding.

In an embodiment, feeding is performed in secondary fermenter, wherein the feeding is exponential feeding.

In another embodiment, feeding is performed in secondary fermenter is constant feeding.

In an embodiment, the constant feeding is about 3 g/L to about 5 g/L in the secondary fermenter.

In an embodiment, feeding is performed in primary and secondary fermenter is different.

In an embodiment, the first exponential feeding is about 10±2% in an incremental order at every hour, wherein the exponential feeding is occurring in the primary fermenter.

In an embodiment, feeding is performed every hour from the beginning of the cell culture.

In an embodiment, the first feeding is started in the primary fermenter after culturing the cells for about 3 hours to 4 hours.

In an embodiment, the feeding of cell culture is performed continuously or intermittently.

In an embodiment, the exponential feeding in the primary fermenter is performed at or about 4 hours after the cell culture.

In an embodiment, the constant feeding is performed in the secondary fermenter from beginning of the cell culture.

In an embodiment, transferring of the cell culture from the primary fermenter to the secondary fermenter occurs once the cell culture of the primary fermenter achieved stationary phase.

In an embodiment, transferring of the cell culture from the primary fermenter to the secondary fermenter occurs once the biomass is about 150 g/L to about 230 g/L.

In an embodiment, transferring of the cell culture from the primary fermenter to the secondary fermenter occurs once the biomass is about 170 g/L to about 200 g/L.

In an embodiment, transferring of the cell culture from the primary fermenter to the secondary fermenter occurs once the cell culture of the primary fermenter achieved OD600 is about 120 to about OD600 140.

In an embodiment, transferring of the cell culture broth from the primary fermenter to the secondary fermenter is about 50% to about 100%.

In an embodiment, the total amount of cell culture broth transferred from the primary fermenter to the secondary fermenter is about 90 to about 95%.

In an embodiment, induction is performed in the secondary fermenter immediately after the cell culture broth has been transferred from the primary fermenter.

In another embodiment, induction is performed in the secondary fermenter for about 8 hours to about 14 hours.

In another embodiment, induction is performed in the secondary fermenter for about 10 hours to about 12 hours.

In an embodiment, induction in the secondary fermenter is performed either in ramp up, ramp down, constant or in bolus mode.

In an embodiment, harvesting the protein from the liquid cell culture, wherein the harvesting occurs after completion of the induction phase.

In an embodiment, the invention provides a cell culture process for production of protein comprising:
a) culturing cells in a primary fermenter;
b) feeding glucose in the primary fermenter under suitable conditions;
c) transferring the cells from the primary fermenter to a secondary fermenter to obtain desired yield of protein of interest; and
d) expressing the protein of interest in the secondary fermenter;
wherein cell density in the primary fermenter and protein titer in the secondary fermenter is increased; wherein the induction is performed in the secondary fermenter to express and harvest the protein of interest from the secondary fermenter.

In an embodiment, induction in the secondary fermenter is performed by using IPTG or arabinose.

In an embodiment, the invention provides a cell culture process for production of protein comprising:
a) culturing cells in a primary fermenter;
b) feeding glucose in the primary fermenter under suitable conditions;
c) transferring the cells from the primary fermenter to a secondary fermenter to obtain desired yield of protein of interest; and
d) expressing the protein of interest in the secondary fermenter;
wherein feeding of glucose is performed at suitable time during the culture in the primary fermenter, increased the cell density in primary fermenter and improved protein titer in secondary fermenter compared to feeding is performed at the beginning of culture in primary fermenter; wherein the induction is performed in secondary fermenter to express and harvest the protein of interest from the secondary fermenter.

In an embodiment, the invention provides a cell culture process for production of protein comprising:
a) culturing cells in a primary fermenter;
b) feeding glucose in the primary fermenter under suitable conditions;
c) transferring the cells from the primary fermenter to a secondary fermenter to obtain desired yield of protein of interest; and
d) expressing the protein of interest in the secondary fermenter;
wherein feeding of glucose is performed at suitable time during the culture in the primary fermenter to achieve cell density at least OD600 120 to OD600 140; wherein the induction is performed in secondary fermenter to express and harvest the protein of interest from the secondary fermenter.

In an embodiment, the feeding of glucose is not performed in the secondary fermenter.

In an embodiment, the feeding of glucose is performed in the primary fermenter after culturing the cells for about 3 hours to about 4 hours.

In an embodiment, the feeding of glucose is performed in the primary fermenter at about 7 hours to about 8 hours when cell culture is performed for about 12 hours.

In an embodiment, the invention provides a cell culture process for production of recombinant protein comprising:
a) culturing cells in a serum free, chemically defined complex media, wherein culturing of cells from a seed culture to a first liquid cell culture;
b) expanding the cells that produce recombinant protein, wherein expanding of cell culture is from the first liquid cell culture to a second liquid cell culture; and
c) harvesting the recombinant protein from the second liquid cell culture;
wherein the first liquid cell culture is transferred to the second liquid cell culture after achieving biomass about 150 g/l to about 230 g/l.

In an embodiment, the invention provides a method for increasing titer of a recombinant protein in a cell culture process comprising:
a) culturing cells in a primary fermenter which produces a desired amount of biomass in cell culture under conditions that allow for protein production;
b) feeding the cells in the primary fermenter with feeding medium containing glucose;
c) transferring the cultured cells from the primary fermenter into a secondary fermenter which is connected to each other; and
d) feeding the cells in the secondary fermenter with feeding medium;
e) expressing the recombinant protein in the secondary fermenter;
wherein transferring of the cultured cells into the secondary fermenter is performed when biomass level is about 150 g/L to about 230 g/L.

In an embodiment, the present invention relates to a process for fermenting cell culture comprising:
a) fermenting cell culture in a first fermenter to form a desired biomass level;
b) introducing the cell culture biomass from the first fermenter to a second fermenter having a second fermentation broth; and
c) introducing a nutrient to at least one of the fermenters.

In an embodiment, the present invention relates to a process for cell culture to produce a recombinant protein, cell culture process comprising:
a) culturing cells in a primary fermenter which produces a desired amount of biomass in cell culture under conditions that allow for protein production;
b) feeding the cells in the primary fermenter with feeding medium containing glucose;
c) transferring the cultured cells from the primary fermenter into a secondary fermenter which is connected to each other; and
d) feeding the cells in the secondary fermenter with feeding medium;
e) expressing the recombinant protein in the secondary fermenter;
wherein transferring of the cultured cells into the secondary fermenter is performed when biomass level is about 150 g/L to about 230 g/L; wherein the cell density, cell biomass, titer and total inclusion bodies are increased.

In an embodiment, the liquid culture can be at least of 200 L, 500 L, 1000 L, 2000 L or more.

In an embodiment, culturing the first liquid cell culture with a seed density is about 3 to 5 OD600 with a variation of 5-10% V/V inoculum.

In an embodiment, the pH is maintained during the cell culture process is from about 6.8 to about 7.2, preferably 7.0.

In an embodiment, the dissolved oxygen (DO) is maintained during the cell culture process is from about 20% to 70%, preferably 30%.

In an embodiment, the temperature is maintained during the cell culture process is from about 35.5° C. to about 38.5° C., preferably 37° C.

In an embodiment, the method discloses the use of primary and secondary fermenter wherein the primary fermenter comprises different cell culture conditions compared to secondary fermenter.

In an embodiment, the inducer used in the secondary fermenter is selected from IPTG and arabinose.

In an embodiment, the cells are cultured for about 8 hours to about 14 hours in the secondary fermenter.

In an embodiment, the cells are cultured for about 10 hours to about 12 hours in the secondary fermenter.

In an embodiment, amount of inclusion bodies obtained from the cell culture is about 15 g/L to about 30 g/L.

In an embodiment, protein titer is obtained from the secondary cell culture is about 4 g/L to about 8 g/L.

In an embodiment, the production process is performed continuously for about 3 days to about 5 days.

In an embodiment, the cell culture media is non-animal origin.

In an embodiment, the cells that produces recombinant proteins in semi continuous large-scale fermenter are microbial cells.

In an embodiment, the cells that produces recombinant proteins in semi continuous large-scale fermenter are bacterial cells.

In an embodiment, the cells that used for producing recombinant proteins in cell culture medium are E. coli cells.

In an embodiment, the recombinant protein produced by E. coli is a fragmented antibody.

In an embodiment, the recombinant protein produced by E. coli is Certolizumab pegol.

In an embodiment, the feeding medium containing glucose is added at a concentration of about 10 g/l to about 20 g/L.

In an embodiment, the secondary fermenter is constantly feeding with glycerol is performed and/or initiated simultaneously keeping the process parameter of secondary fermenter same as of primary fermenter.

In an embodiment, a cell culture process for production of a recombinant protein, comprising culturing the cells in primary and secondary fermenter and/or bioreactor wherein the primary and secondary fermenter and/or bioreactor is connected to each other to perform transfer of cultured cells from primary fermenter and/or bioreactor to secondary fermenter and/or bioreactor.

In an embodiment, a cell culture process for production of a recombinant protein, comprising culturing the cells in primary fermenter and/or bioreactor to obtain suitable cell biomass and transferred the cell biomass to secondary fermenter and/or bioreactor for protein production.

In some embodiments, a cell culture process for production of a recombinant protein, comprising:
a) culturing cells in a primary fermenter which produces a desired amount of biomass in cell culture under conditions that allow for protein production;
b) feeding the cells in the primary fermenter with feeding medium containing glucose;
c) transferring the cultured cells from the primary fermenter into a secondary fermenter which is connected to each other; and
d) feeding the cells in the secondary fermenter with feeding medium;
e) expressing the recombinant protein in the secondary fermenter;
wherein transferring of the cultured cells into the secondary fermenter is performed when biomass level is about 150 g/L to about 230 g/L.

In some embodiments, the feeding in the secondary fermenter is started from the beginning of the cell culture.

In some embodiments, the feeding is about 3 g/L to about 5 g/L in the secondary fermenter.

In some embodiments, the transferring of the cell culture from the primary fermenter to the secondary fermenter occurs once the cell culture of the primary fermenter achieved about OD600 120 to about OD600 140.

In some embodiments, transferring of the cell culture broth from the primary fermenter to the secondary fermenter is about 50% to about 100%.

In some embodiments, induction is performed in the secondary fermenter by using inducing agent IPTG or arabinose for about 10 hours to about 12 hours.

In some embodiments, the recombinant protein produced by E. coli is a fragmented antibody.

In some embodiments, the fragmented antibody is Certolizumab pegol.

In some embodiments, induction in the secondary fermenter is performed either in ramp up, ramp down, constant or in bolus mode.

In some embodiments, a cell culture process for production of protein comprising:
a) culturing cells in a primary fermenter;
b) feeding glucose in the primary fermenter under suitable conditions;
c) transferring the cells from the primary fermenter to a secondary fermenter to obtain desired yield of protein of interest; and
d) expressing the protein of interest in the secondary fermenter;
wherein cell density in the primary fermenter and protein titer in the secondary fermenter is increased; wherein the induction is performed in the secondary fermenter to express and harvest the protein of interest from the secondary fermenter.

In some embodiments, the feeding in the secondary fermenter is started from the beginning of the cell culture.

In some embodiments, the feeding is about 3 g/L to about 5 g/L in the secondary fermenter.

In some embodiments, the transferring of the cell culture from the primary fermenter to the secondary fermenter occurs once the cell culture of the primary fermenter achieved about OD600 120 to about OD600 140.

In some embodiments, the transferring of the cell culture broth from the primary fermenter to the secondary fermenter is about 50% to about 100%.

In some embodiments, induction is performed in the secondary fermenter by using inducing agent IPTG or arabinose for about 10 hours to about 12 hours.

In some embodiments, the recombinant protein produced by E. coli is a fragmented antibody.

In some embodiments, the fragmented antibody is Certolizumab pegol.

In some embodiments, induction in the secondary fermenter is performed either in ramp up, ramp down, constant or in bolus mode.

In some embodiments, a cell culture process for production of recombinant protein comprising:
a) culturing cells in a serum free, chemically defined complex media, wherein culturing of cells from a seed culture to a first liquid cell culture;
b) expanding the cells that produce recombinant protein, wherein expanding of the cell culture from the first liquid cell culture to a second liquid cell culture; and
c) harvesting the recombinant protein from the second liquid cell culture;
wherein the first liquid cell culture is transferred to the second liquid cell culture after achieving biomass about 150 g/l to about 230 g/l.

In some embodiments, a cell culture process for production of protein comprising:
a) culturing cells in a primary fermenter;
b) feeding glucose in the primary fermenter under suitable conditions;
c) transferring the cells from the primary fermenter to a secondary fermenter to obtain desired yield of protein of interest; and
d) expressing the protein of interest in the secondary fermenter;
wherein feeding of glucose is performed at suitable time during the culture in the primary fermenter to achieve cell density at least OD600 120 to OD600 140; wherein the induction is performed in secondary fermenter to express and harvest the protein of interest from the secondary fermenter.

In some embodiments, method for increasing productivity of a cell culture process comprising:
a) culturing cells in a primary fermenter which produces a desired amount of biomass in cell culture under conditions that allow for protein production;
b) feeding the cells in the primary fermenter with feeding medium containing glucose;
c) transferring the cultured cells from the primary fermenter into a secondary fermenter which is connected to each other; and
d) feeding the cells in the secondary fermenter with feeding medium;
e) expressing the recombinant protein in the secondary fermenter;
wherein transferring of the cultured cells into the secondary fermenter is performed when biomass level is about 150 g/L to about 230 g/L.

In some embodiments, method for increasing titer of a recombinant protein in a cell culture process comprising:
a) culturing cells in a primary fermenter which produces a desired amount of biomass in cell culture under conditions that allow for protein production;
b) feeding the cells in the primary fermenter with feeding medium containing glucose;
c) transferring the cultured cells from the primary fermenter into a secondary fermenter which is connected to each other; and
d) feeding the cells in the secondary fermenter with feeding medium;
e) expressing the recombinant protein in the secondary fermenter;
wherein transferring of the cultured cells into the secondary fermenter is performed when biomass level is about 150 g/L to about 230 g/L.

In some embodiments, the feeding in the secondary fermenter is started from the beginning of the cell culture.

In some embodiments, the feeding is about 3 g/L to about 5 g/L in the secondary fermenter.

In some embodiments, the transferring of the cell culture from the primary fermenter to the secondary fermenter occurs once the cell culture of the primary fermenter achieved about OD600 120 to about OD600 140.

In some embodiments, the transferring of the cell culture broth from the primary fermenter to the secondary fermenter is about 50% to about 100%.

In some embodiments, induction is performed in the secondary fermenter by using inducing agent IPTG or arabinose for about 10 hours to about 12 hours.

In some embodiments, the recombinant protein produced by E. coli is a fragmented antibody.

In some embodiments, the fragmented antibody is Certolizumab pegol.

In some embodiments, induction in the secondary fermenter is performed either in ramp up, ramp down, constant or in bolus mode.

In some embodiments, a process for cell culture to produce a recombinant protein, cell culture process comprising:
a) culturing cells in a primary fermenter which produces a desired amount of biomass in cell culture under conditions that allow for protein production;
b) feeding the cells in the primary fermenter with feeding medium containing glucose;
c) transferring the cultured cells from the primary fermenter into a secondary fermenter which is connected to each other; and
d) feeding the cells in the secondary fermenter with feeding medium;
e) expressing the recombinant protein in the secondary fermenter;
wherein transferring of the cultured cells into the secondary fermenter is performed when biomass level is about 150 g/L to about 230 g/L; wherein the cell density, cell biomass, titer and total inclusion bodies are increased.

In some embodiments, the titer is at least from about 5 g/L to about 7 g/L.

In some embodiments, the titer is at least from about 6 g/L to about 7 g/L.

In some embodiments, the biomass is about 3000 g to about 6000 g.

In some embodiments, the biomass is at least about 4000 g to about 5500 g.

In some embodiments, the feeding in the secondary fermenter is started from the beginning of the cell culture.

In some embodiments, the feeding is about 3 g/L to about 5 g/L in the secondary fermenter.

In some embodiments, the transferring of the cell culture from the primary fermenter to the secondary fermenter occurs once the cell culture of the primary fermenter achieved about OD600 120 to about OD600 140.

In some embodiments, the transferring of the cell culture broth from the primary fermenter to the secondary fermenter is about 50% to about 100%.

In some embodiments, induction is performed in the secondary fermenter by using inducing agent IPTG or arabinose for about 10 hours to about 12 hours.

In some embodiments, the recombinant protein produced by E. coli is a fragmented antibody.

In some embodiments, the fragmented antibody is Certolizumab pegol.

In some embodiments, induction in the secondary fermenter is performed either in ramp up, ramp down, constant or in bolus mode.

The present invention provides an example for illustration purpose which should not be considered to limit the scope of the present invention with the described examples.

### Example 1

### Conventional Single Fermenter Process:

Seed 1 developed with clone BL21AI for about 12 to about 18 hours in LB media (Make: Hi Media). At culture OD600 of 3 to 5, the Seed 1 culture is transferred to another Shake flask to develop the seed II culture. Seed II shake flask incubated at 37 deg C. for 4-6 hours to achieve OD600 of 5-7 for fermenter inoculation. A fermenter (Sartorius Stedim, 10 L) containing sterilized batch media (nearly 60% of fermenter working volume) inoculated with seed II culture with 10% V/V. Post inoculation od600 monitored hourly and Feed initiated with glucose at a concentration of 10 g/l to 20 g/l is added either from the after 3 hours to 4 hours as per the carbon content in batch media. The fermenter pH is maintained at 7±0.4, DO is maintained at 30% which varies from 20% to 100%, temperature is maintained at 37±2° C.

The culture grown till OD600120 at nearly 12 hours and the fermenter induction initiated with (L)+ arabinose 50 mM, and constant feeding of glycerol is also initiated simultaneously. The protein expression is monitored at 4^{th} hour, 8^{th} hour of induction. After completion of 10 h hour induction, batch is harvested.

The biomass recovered from harvested broth using centrifugation (Beckman coulter) 7000 RPM for 15 minutes at 5 deg C. A cell suspension prepared using cell lysis buffer (100 MM Tris and 1 mM DTT) lysis is performed mechanically using high pressure homogenizer (niro soavi) at 700-900 bar, 5° C., 3 passes and inclusion bodies recovered by centrifugation at 16000 RCF for 30 minutes at 5 deg C. The inclusion bodies are washed using buffers (Tris 100 mM, NaCl 0.5 g/l and EDTA 5 mM) and final wash with 50 mM Tris to remove cell debris and final inclusion bodies recovered.

The inclusion bodies recovered is about 23 g/l of harvest broth and the IB's are transferred to downstream for further purification steps.

### Results

TABLE 1
Conventional (Single Fermenter) process.

| Process Step | Outputs |
|---|---|
| Harvest OD600 | 126 |
| Harvest volume (mL) | 9500 |
| Biomass (g) | 972 |
| Inclusion bodies (g) | 251 |
| Titer (g/L) by RP-HPLC | 5.9 |

### Example 2

### Semi Continuous Process:

Seed 1 developed with clone BL21AI for about 12 to about 18 hours in LB media (Make: Hi Media). At culture OD600 of 3 to 5, the Seed 1 culture is transferred to another Shake flask to develop the seed II culture. Seed II shake flask incubated at 37° C. for 4-6 hours to achieve OD600 of 5-7 for fermenter inoculation. A fermenter (Sartorius Stedim, 10 L) containing sterilized batch media (nearly 60% of fermenter working volume) inoculated with seed II culture with 10% V/V. Post inoculation OD600 monitored hourly, and Feed initiated with glucose at a concentration of 10 g/l to 20 g/l is added after 3 hours to 4 hours as per the carbon content in batch media. The fermenter pH is maintained at 7±0.4, DO is maintained at 30% which varies from 20% to 100%, temperature is maintained at 37±2° C. The culture grown till OD600120 at nearly 12 hours and 90% to 95% primary fermenter cell culture broth with OD600 120 is transferred to the secondary fermenter (Sartorius Stedim, 10 L) and remaining 5% to 10% of primary cell culture in the primary fermenter is supplemented with next lot of batch media. Just after transfer of the primary cell culture broth to secondary fermenter, induction is initiated with (L)+ arabinose 50 mM, and constant feeding of glycerol is initiated simultaneously keeping the process parameter of secondary fermenter same as of primary fermenter. Post completion of 10 hours induction, culture withdrawn aseptically. Again 90% to 95% of cell culture from primary fermenter is transferred to the secondary fermenter and induction & feeding are initiated as explained. This process is repeated till four more passages. The biomass recovered from harvested broth using centrifugation (Beckman coulter) 7000 RPM for 15 minutes at 5 deg C. A cell suspension prepared using cell lysis buffer (100 MM Tris and 1 mM DTT) lysis is performed mechanically using high pressure homogenizer (niro soavi) at 700-900 bar, 5° C., 3 passes and inclusion bodies recovered by centrifugation at 16000 RCF for 30 minutes at 5 deg C. The inclusion bodies are washed using buffers (Tris 100 mM, NaCl 0.5 g/l and EDTA 5 mM) and final wash with 50 mM Tris to remove cell debris and final inclusion bodies recovered. The inclusion bodies recovered is about 25 g/l of harvest broth and the IB's are transferred to downstream for further purification steps.

### Results

TABLE 2
Semi continuous process.

| Process Steps | Output |
|---|---|
| Harvest OD600 range | 118-135 |
| Harvest (L) | (10*5) |
| Biomass (g) | 4976 |
| Inclusion bodies (g) | 1250 |
| Titer (g/L) | 6.8 |

Table 2 semi continuous process shows higher productivity in terms of biomass, inclusion bodies and titer in a comparatively shorter time which is equivalent to 5 times of the conventional process as described in example 1.

1. A cell culture process for production of a recombinant protein, comprising culturing the cells in primary and secondary fermenter and/or bioreactor wherein the primary and secondary fermenter and/or bioreactor is connected to each other to perform transfer of cultured cells from primary fermenter and/or bioreactor to secondary fermenter and/or bioreactor.

2. A cell culture process for production of a recombinant protein, comprising culturing the cells in primary fermenter and/or bioreactor to obtain suitable cell biomass and transferred the cell biomass to secondary fermenter and/or bioreactor for protein production.

3. The process as claimed in claim 2 wherein the cell biomass about 100 g/L to about 230 g/L.

4. The process as claimed in claim 2 wherein the cell biomass about 150 g/L to about 230 g/L.

5. A cell culture process for production of a recombinant protein, comprising:
a) culturing cells in a primary fermenter and/or bioreactor which produces a desired amount of biomass in cell culture;
b) feeding the cells in the primary fermenter with feeding medium containing glucose;
c) transferring the cultured cells from the primary fermenter into a secondary fermenter which is connected to each other; and
d) feeding the cells in the secondary fermenter with feeding medium;
e) expressing the recombinant protein in the secondary fermenter;
wherein transferring of the cultured cells into the secondary fermenter is performed when biomass level is about 150 g/L to about 230 g/L.

6. The process as claimed in claim 5, wherein the feeding in the secondary fermenter is started from the beginning of the cell culture.

7. The process as claimed in claim 5, wherein the feeding is about 3 g/L to about 5 g/L in the secondary fermenter.

8. The process as claimed in claim 5, wherein the transferring of the cell culture from the primary fermenter to the secondary fermenter occurs once the cell culture of the primary fermenter achieved about OD600 120 to about OD600 140.

9. The process as claimed in claim 5, wherein the transferring of the cell culture broth from the primary fermenter to the secondary fermenter is about 50% to about 100%.

10. The process as claimed in claim 5, wherein induction is performed in the secondary fermenter by using inducing agent IPTG or arabinose for about 10 hours to about 12 hours.

11. The process as claimed in claim 5, wherein the recombinant protein produced by E. coli is a fragmented antibody.

12. The process as claimed in claim 5, wherein the fragmented antibody is Certolizumab pegol.

13. The process as claimed in claim 1 or claim 2 or claim 5, wherein induction in the secondary fermenter is performed either in ramp up, ramp down, constant or in bolus mode.

14. A cell culture process for production of protein comprising:
a) culturing cells in a primary fermenter;
b) feeding glucose in the primary fermenter under suitable conditions;
c) transferring the cells from the primary fermenter to a secondary fermenter to obtain desired yield of protein of interest; and
d) expressing the protein of interest in the secondary fermenter;
wherein cell density in the primary fermenter and protein titer in the secondary fermenter is increased; wherein the induction is performed in the secondary fermenter to express and harvest the protein of interest from the secondary fermenter.

15. The process as claimed in claim 14, wherein the feeding in the secondary fermenter is started from the beginning of the cell culture.

16. The process as claimed in claim 14, wherein the feeding is about 3 g/L to about 5 g/L in the secondary fermenter.

17. The process as claimed in claim 14, wherein the transferring of the cell culture from the primary fermenter to the secondary fermenter occurs once the cell culture of the primary fermenter achieved about OD600 120 to about OD600 140.

18. The process as claimed in claim 14, wherein the transferring of the cell culture broth from the primary fermenter to the secondary fermenter is about 50% to about 100%.

19. The process as claimed in claim 14, wherein induction is performed in the secondary fermenter by using inducing agent IPTG or arabinose for about 10 hours to about 12 hours.

20. The process as claimed in claim 14, wherein the recombinant protein produced by E. coli is a fragmented antibody.

21. The process as claimed in claim 14, wherein the fragmented antibody is Certolizumab pegol.

22. The process as claimed in claim 14, wherein induction in the secondary fermenter is performed either in ramp up, ramp down, constant or in bolus mode.

23. A cell culture process for production of recombinant protein comprising:
a) culturing cells in a serum free, chemically defined complex media, wherein culturing of cells from a seed culture to a first liquid cell culture;
b) expanding the cells that produce recombinant protein, wherein expanding of the cell culture from the first liquid cell culture to a second liquid cell culture; and
c) harvesting the recombinant protein from the second liquid cell culture;
wherein the first liquid cell culture is transferred to the second liquid cell culture after achieving biomass about 150 g/l to about 230 g/l.

24. A cell culture process for production of protein comprising:
a) culturing cells in a primary fermenter;
b) feeding glucose in the primary fermenter under suitable conditions;
c) transferring the cells from the primary fermenter to a secondary fermenter to obtain desired yield of protein of interest; and
d) expressing the protein of interest in the secondary fermenter;
wherein feeding of glucose is performed at suitable time during the culture in the primary fermenter to achieve cell density at least OD600 120 to OD600 140; wherein the induction is performed in secondary fermenter to express and harvest the protein of interest from the secondary fermenter.

25. A method for increasing productivity of a cell culture process comprising:
a) culturing cells in a primary fermenter which produces a desired amount of biomass in cell culture under conditions that allow for protein production;
b) feeding the cells in the primary fermenter with feeding medium containing glucose;
c) transferring the cultured cells from the primary fermenter into a secondary fermenter which is connected to each other; and
d) feeding the cells in the secondary fermenter with feeding medium;
e) expressing the recombinant protein in the secondary fermenter;
wherein transferring of the cultured cells into the secondary fermenter is performed when biomass level is about 150 g/L to about 230 g/L.

26. A method for increasing titer of a recombinant protein in a cell culture process comprising:
a) culturing cells in a primary fermenter which produces a desired amount of biomass in cell culture under conditions that allow for protein production;
b) feeding the cells in the primary fermenter with feeding medium containing glucose;
c) transferring the cultured cells from the primary fermenter into a secondary fermenter which is connected to each other; and
d) feeding the cells in the secondary fermenter with feeding medium;
e) expressing the recombinant protein in the secondary fermenter;
wherein transferring of the cultured cells into the secondary fermenter is performed when biomass level is about 150 g/L to about 230 g/L.

27. The process as claimed in claim 26, wherein the feeding in the secondary fermenter is started from the beginning of the cell culture.

28. The process as claimed in claim 26, wherein the feeding is about 3 g/L to about 5 g/L in the secondary fermenter.

29. The process as claimed in claim 26, wherein the transferring of the cell culture from the primary fermenter to the secondary fermenter occurs once the cell culture of the primary fermenter achieved about OD600 120 to about OD600 140.

30. The process as claimed in claim 26, wherein the transferring of the cell culture broth from the primary fermenter to the secondary fermenter is about 50% to about 100%.

31. The process as claimed in claim 26, wherein induction is performed in the secondary fermenter by using inducing agent IPTG or arabinose for about 10 hours to about 12 hours.

32. The process as claimed in claim 26, wherein the recombinant protein produced by E. coli is a fragmented antibody.

33. The process as claimed in claim 26, wherein the fragmented antibody is Certolizumab pegol.

34. The process as claimed in claim 26, wherein induction in the secondary fermenter is performed either in ramp up, ramp down, constant or in bolus mode.

35. A process for cell culture to produce a recombinant protein, cell culture process comprising:
a) culturing cells in a primary fermenter which produces a desired amount of biomass in cell culture under conditions that allow for protein production;
b) feeding the cells in the primary fermenter with feeding medium containing glucose;
c) transferring the cultured cells from the primary fermenter into a secondary fermenter which is connected to each other; and
d) feeding the cells in the secondary fermenter with feeding medium;
e) expressing the recombinant protein in the secondary fermenter;
wherein transferring of the cultured cells into the secondary fermenter is performed when biomass level is about 150 g/L to about 230 g/L; wherein the cell density, cell biomass, titer and total inclusion bodies are increased.

36. The process as claimed in claim 35, wherein the titer is at least from about 5 g/L to about 7 g/L.

37. The process as claimed in claim 35, wherein the titer is at least from about 6 g/L to about 7 g/L.

38. The process as claimed in claim 35, wherein the biomass is about 3000 g to about 6000 g.

39. The process as claimed in claim 35, wherein the biomass is at least about 4000 g to about 5500 g.

40. The process as claimed in claim 35, wherein the feeding in the secondary fermenter is started from the beginning of the cell culture.

41. The process as claimed in claim 35, wherein the feeding is about 3 g/L to about 5 g/L in the secondary fermenter.

42. The process as claimed in claim 35, wherein the transferring of the cell culture from the primary fermenter to the secondary fermenter occurs once the cell culture of the primary fermenter achieved about OD600 120 to about OD600 140.

43. The process as claimed in claim 35, wherein the transferring of the cell culture broth from the primary fermenter to the secondary fermenter is about 50% to about 100%.

44. The process as claimed in claim 35, wherein induction is performed in the secondary fermenter by using inducing agent IPTG or arabinose for about 10 hours to about 12 hours.

45. The process as claimed in claim 35, wherein the recombinant protein produced by E. coli is a fragmented antibody.

46. The process as claimed in claim 35, wherein the fragmented antibody is Certolizumab pegol.

47. The process as claimed in claim 35, wherein induction in the secondary fermenter is performed either in ramp up, ramp down, constant or in bolus mode.
